(19) United States
(12) Patent Application Publication
(10) Pub. No.: US 2025/0193346 A1
(43) Pub. Date: Jun. 12, 2025

(54) IMAGE PROCESSING CONTROL SYSTEM, IMAGE PROCESSING METHOD, AND PROGRAM

(71) Applicant: SONY GROUP CORPORATION, TOKYO (JP)

(72) Inventor: TETSUYA MITANI, TOKYO (JP)

(21) Appl. No.: 18/843,346

(22) PCT Filed: Feb. 22, 2023
(86) PCT No.: PCT/JP2023/006372

§ 371 Date: Sep. 3, 2024

(30) Foreign Application Priority Data
Mar. 10, 2022 (JP) 2022-037135

(51) Int. Cl. H04N 7/18; A61B 1/00; A61B 6/46; A61B 6/50; H04N 5/262; H04N 23/50

(52) U.S. Cl. CPC H04N 7/181; A61B 1/000095; A61B 6/463; A61B 6/465; A61B 6/504; H04N 5/2628; H04N 23/555

(57) ABSTRACT

The present disclosure relates to an image processing control system, an image processing method, and a program that enable easy application of image processing settings suitable for medical images. An image processing control system includes a plurality of medical video sources connected via a network, a plurality of output sources connected via a network, a switching unit that switches an output destination of the medical video sources to one of a plurality of the output sources, and an image processing control unit that controls an image processing setting for performing display of an image on which image processing suitable for a combination of the medical video sources and the output sources has been applied even when the output destination of the medical video sources is switched. The present technology can be applied to, for example, an operating room integration system that integrally manages a plurality of medical devices, medical videos, and the like.

### TECHNICAL FIELD

The present disclosure relates to an image processing control system, an image processing method, and a program, and more particularly relates to an image processing control system, an image processing method, and a program capable of easily applying image processing settings suitable for medical images.

### BACKGROUND ART

In recent years, an operating room provided with an operating room integration system that integrally manages a plurality of medical devices, medical videos, and the like is increasing.

For example, Patent Document 1 discloses a technique in which a medical staff in an operating room uses a controller to control, for example, which medical device's video is displayed on which monitor, and control settings of respective medical devices and monitors, thereby executing optimal settings on the basis of respective settings of the medical devices and the monitors.

### CITATION LIST

### Patent Document

Patent Document 1: WO 2019/176556 A1

### SUMMARY OF THE INVENTION

### Problems to be Solved by the Invention

However, conventionally, it is troublesome to perform detailed settings corresponding to each of the medical devices and medical images, and it is required that these settings are easily applied.

The present disclosure has been made in view of such a situation, and is to enable image processing setting suitable for medical images to be easily applied.

### Solutions to Problems

An image processing control system according to one aspect of the present disclosure includes a plurality of medical video sources connected via a network, a plurality of output sources connected via a network, a switching unit that switches an output destination of the medical video sources to one of a plurality of the output sources, and an image processing control unit that controls an image processing setting for performing display of an image on which image processing suitable for a combination of the medical video sources and the output sources has been applied even when the output destination of the medical video sources is switched.

An image processing method or a program according to one aspect of the present disclosure includes controlling, even when an output destination of a plurality of medical video sources connected via a network is switched to one of a plurality of output sources connected via the network, an image processing setting for performing display of an image on which image processing suitable for a combination of the plurality of medical video sources and the plurality of output sources connected has been performed.

In one aspect of the present disclosure, even when an output destination of a plurality of medical video sources connected via a network is switched to one of a plurality of output sources connected via the network, an image processing setting for performing display of an image on which image processing suitable for a combination of the plurality of medical video sources and the plurality of output sources connected has been performed is controlled.

### BRIEF DESCRIPTION OF DRAWINGS

FIG. 1 is a block diagram illustrating a configuration example of an embodiment of a medical image processing control system to which the present technology is applied.

FIG. 2 is a diagram illustrating an example of a setting table.

FIG. 3 is a diagram illustrating an example of a control screen.

FIG. 4 is a flowchart describing image processing setting processing.

FIG. 5 is a block diagram illustrating a configuration example of an embodiment of a computer to which the present technology is applied.

### MODE FOR CARRYING OUT THE INVENTION

Hereinafter, specific embodiments according to the present technology will be described in detail with reference to the drawings.

### <Configuration Example of Medical Image Processing Control System>

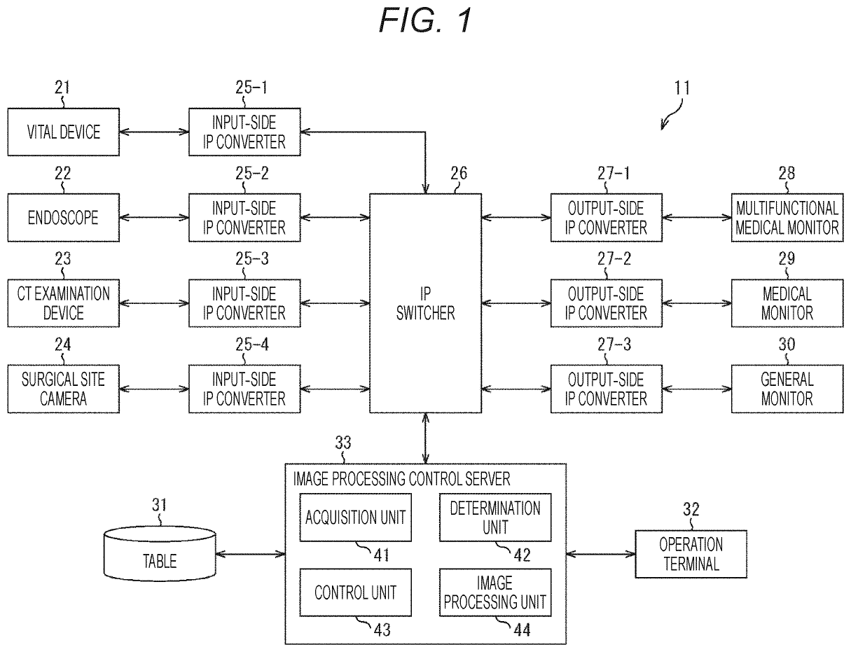

FIG. 1 is a block diagram illustrating a configuration example of an embodiment of a medical image processing control system to which the present technology is applied.

A medical image processing control system 11 illustrated in FIG. 1 is configured by connecting a plurality of medical video sources and a plurality of output sources on an Internet Protocol (IP) network, and can control image processing for various medical images (moving images or still images). In the example illustrated in FIG. 1, the medical image processing control system 11 includes an endoscope 22, a computed tomography (CT) examination device 23, and a surgical site camera 24 as medical video sources, and includes a multifunctional medical monitor 28, a medical monitor 29, and a general monitor 30 as output sources. Moreover, the medical image processing control system 11 includes a vital device 21, input-side IP converters 25-1 to 25-4, output-side IP converters 27-1 to 27-3, a storage unit 31, an operation terminal 32, and an image processing control server 33.

The vital device 21, the endoscope 22, the CT examination device 23, and the surgical site camera 24 are connected to an IP switcher 26 via the input-side IP converters 25-1 to 25-4, respectively. The multifunctional medical monitor 28, the medical monitor 29, and the general monitor 30 are connected to the IP switcher 26 via the output-side IP converters 27-1 to 27-3, respectively. Furthermore, the image processing control server 33 is connected to the IP switcher 26, and the storage unit 31 and the operation terminal 32 are connected to the image processing control server 33.

The vital device 21 acquires patient vital information such as pulse, blood pressure, respiration, and body temperature, for example, and outputs an image of a vital screen (hereinafter referred to as a vital image) in which the vital information is arranged side by side on one screen. Then, the image of the vital image output from the vital device 21 is subjected to IP conversion by the input-side IP converter 25-1 and then input to the IP switcher 26.

The endoscope 22 outputs, for example, an image (hereinafter referred to as an endoscopic image) obtained by capturing a state of the inside of the body of the patient, such as the esophagus and the gastrointestinal tract. Then, the endoscopic image output from the endoscope 22 is subjected to IP conversion by the input-side IP converter 25-2 and then input to the IP switcher 26.

For example, the CT examination device 23 outputs an image (hereinafter referred to as a CT image) obtained by cross-sectional imaging of the inside of the patient's body using X-rays. Then, the CT image output from the CT examination device 23 is subjected to IP conversion by the input-side IP converter 25-3 and then input to the IP switcher 26.

The surgical site camera 24 outputs, for example, an image obtained by imaging a state in an operating room in which a patient is operated (hereinafter referred to as a surgical site image). Then, the surgical site image output from the surgical site camera 24 is subjected to IP conversion by the input-side IP converter 25-4 and then input to the IP switcher 26.

The IP switcher 26 switches output destinations of the vital image, the endoscopic image, the CT image, and the surgical site image supplied via the input-side IP converters 25-1 to 25-4 to one of the output-side IP converters 27-1 to 27-3, and outputs the respective images. That is, the IP switcher 26 can switch connections between a plurality of medical video sources and a plurality of output sources.

The multifunctional medical monitor 28 displays an image supplied from the IP switcher 26 via the output-side IP converter 27-1 and IP-converted by the output-side IP converter 27-1 among the vital image, the endoscopic image, the CT image, and the surgical site image. Furthermore, the multifunctional medical monitor 28 has an image processing function such as, for example, image processing for emphasizing display of a blood vessel, image processing for performing gamma correction suitable for medical images, and the like.

The medical monitor 29 displays an image supplied from the IP switcher 26 via the output-side IP converter 27-2 and IP-converted by the output-side IP converter 27-2 among the vital image, the endoscopic image, the CT image, and the surgical site image. Furthermore, the medical monitor 29 has an image processing function such as a sharpening filter and a luminance enhancement filter suitable for medical images, for example.

The general monitor 30 displays an image supplied from the IP switcher 26 via the output-side IP converter 27-3 and IP-converted by the output-side IP converter 27-3 among the vital image, the endoscopic image, the CT image, and the surgical site image. Note that the general monitor 30 does not have an image processing function suitable for medical images.

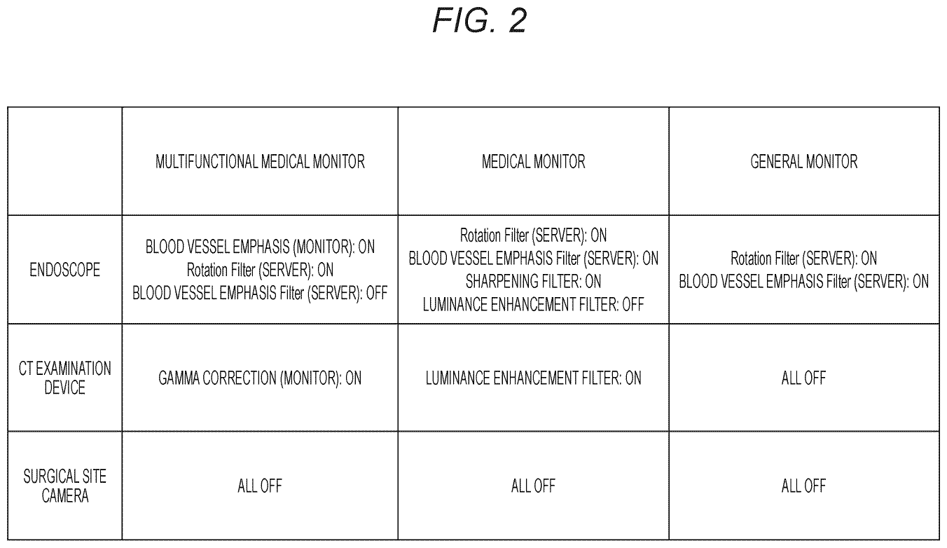

The storage unit 31 stores a setting table (see FIG. 2) in which setting information of image processing applied in each combination is registered for each combination of a plurality of medical video sources and a plurality of output sources.

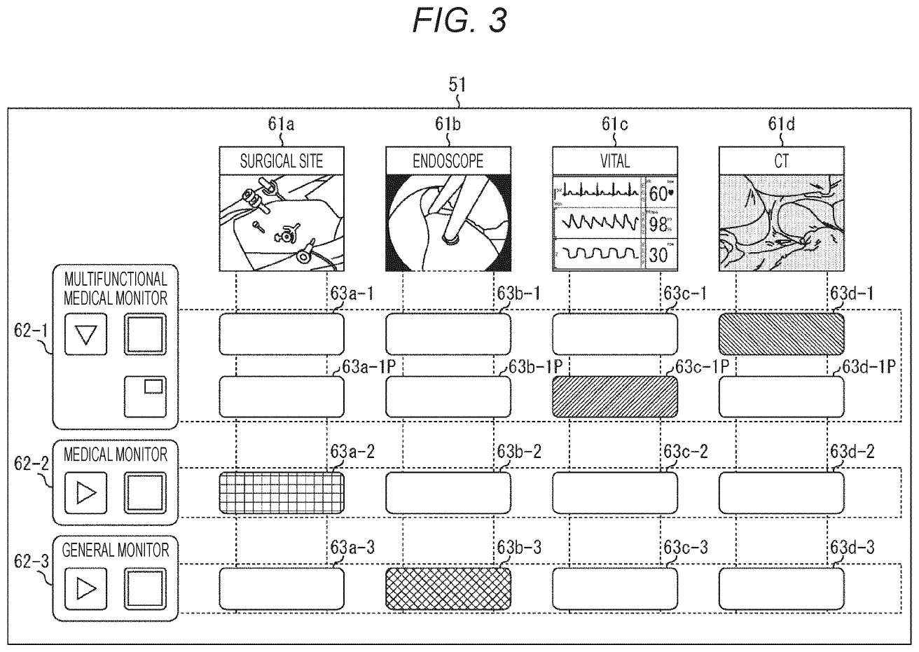

The operation terminal 32 includes, for example, a touch panel, and displays a control screen (see FIG. 3) for switching the connection between the plurality of medical video sources and the plurality of output sources on the touch panel. Then, the operation terminal 32 acquires a touch operation on the control screen by the user, and supplies operation information indicating the operation content to the image processing control server 33.

According to the operation information supplied from the operation terminal 32, the image processing control server 33 can refer to the setting table stored in the storage unit 31, and can control image processing settings for performing display of an image on which image processing suitable for a combination of a medical video source and an output source has been applied. As illustrated, the image processing control server 33 includes an acquisition unit 41, a determination unit 42, a control unit 43, and an image processing unit 44.

The acquisition unit 41 acquires information of a medical video source and information of an output source included in the medical image processing control system 11 via the IP switcher 26, and supplies these pieces of information to the determination unit 42.

The determination unit 42 refers to the setting table stored in the storage unit 31 on the basis of the information supplied from the acquisition unit 41, and searches for setting information of the combination of the medical video source and the output source. Then, the determination unit 42 determines whether or not the setting information of the corresponding combination is registered in the setting table, and in a case where it is determined that the setting information of the corresponding combination is registered in the setting table, the determination unit notifies the control unit 43 that the setting information is registered.

The control unit 43 reads from the setting table the setting information notified from the determination unit 42. Then, the control unit 43 controls the setting (on/off) of the image processing to be applied as a whole of the medical image processing control system 11 according to the setting information for each combination of the medical video source and the output source.

The image processing unit 44 includes image processing provided in the image processing control server 33, for example, image processing functions such as a rotation filter and a blood vessel emphasis filter suitable for medical images. Then, the image processing unit 44 turns on/off the image processing under the control of the control unit 43.

The medical image processing control system 11 is configured in this manner, and even when the output destination of the medical video source is switched to any of the plurality of output sources, it is possible to easily perform an image processing setting so that an image suitable for a combination thereof is displayed.

For example, in the medical image processing control system 11, in a case where the information (metadata) of the CT image output from the CT examination device 23 is described as the information of the medical video source acquired by the acquisition unit 41, when the output destination is the multifunctional medical monitor 28, an image processing setting for turning on the gamma correction of the multifunctional medical monitor 28 is performed. Note that, in a case where the medical image processing control system 11 has an image analysis function, which medical video source it is may be estimated by analyzing an image output from the medical video source instead of using the information of the medical video source. Furthermore, the medical image processing control system 11 may acquire the information of the medical video source by the user operating the operation terminal 32 to designate which medical video source it is, for example, by selecting the medical video source from a category prepared in advance.

Furthermore, in the medical image processing control system 11, for example, the acquisition unit 41 can acquire information of an output source (for example, standard information such as characteristic data) according to the standard of High-Definition Multimedia Interface (HDMI) (registered trademark). Then, the medical image processing control system 11 can recognize what function the output source has if the output source is of a known type. For example, in a case where the medical image processing control system 11 recognizes that the output source is the medical monitor 29 having an image processing function such as a sharpening filter and a luminance enhancement filter, when an endoscopic image is output to the medical monitor 29, an image processing setting for turning on the image processing function such as a sharpening filter and a luminance enhancement filter is performed in the output source.

Furthermore, in the medical image processing control system 11, in a case where it can be analyzed that the image output from the medical video source is rotating, that is, in a case of an endoscopic image, an image processing setting for turning on the rotation filter by the image processing unit 44 is performed. Then, in a case where the output source that outputs the image is switched, the medical image processing control system 11 can continuously apply the rotation filter to the output source after the switching.

FIG. 2 is a diagram illustrating an example of a setting table stored in the storage unit 31.

For example, in the setting table, for the combination of the endoscope 22 and the multifunctional medical monitor 28, setting information for turning on image processing for emphasizing the display of blood vessels in the multifunctional medical monitor 28, turning on the rotation filter of the image processing unit 44, and turning off the blood vessel emphasis filter of the image processing unit 44 is registered. Further, for the combination of the endoscope 22 and the medical monitor 29, setting information for turning on the rotation filter and the blood vessel emphasis filter of the image processing unit 44, turning on the sharpening filter of the medical monitor 29, and turning off the luminance enhancement filter of the medical monitor 29 is registered. Furthermore, for the combination of the endoscope 22 and the general monitor 30, setting information for turning on the rotation filter and the blood vessel emphasis filter of the image processing unit 44 is registered.

Further, in the setting table, for the combination of the CT examination device 23 and the multifunctional medical monitor 28, setting information for turning on the gamma correction of the multifunctional medical monitor 28 is registered. Furthermore, setting information for turning on the luminance enhancement filter of the medical monitor 29 is registered for the combination of the CT examination device 23 and the medical monitor 29. Further, for the combination of the CT examination device 23 and the general monitor 30, setting information for turning off all pieces of image processing is registered. Moreover, in the setting table, for the surgical site camera 24, setting information for turning off all pieces of image processing is registered for each combination with the multifunctional medical monitor 28, the medical monitor 29, and the general monitor 30.

With reference to such a setting table, in the medical image processing control system 11, the image processing setting is controlled in the image processing control server 33.

For example, in the medical image processing control system 11, in a case where the medical video source is the endoscope 22 and an endoscopic image is output to an output source that does not have a function such as rotation correction, the rotation filter of the image processing unit 44 is turned on. Here, the rotation filter is a correction filter for correcting the rotation of the endoscopic image, and for example, the image processing unit 44 calculates a rotation angle, and the output-side IP converter 27 can perform the rotation correction according to the rotation angle.

Furthermore, in the medical image processing control system 11, the setting on the output source side may be changed. For example, in a case where both the multifunctional medical monitor 28 and the image processing unit 44 have an image processing function of emphasizing the display of the blood vessel, the multifunctional medical monitor 28 side may be turned on, the image processing unit 44 side may be turned off, the multifunctional medical monitor 28 side may be turned off, and the image processing unit 44 may be turned on. That is, in a case where image processing for emphasizing the display of the blood vessel of the both is turned on, it is assumed that the display of the blood vessel is excessively emphasized, and thus it is preferable to turn off either one of the image processing and the image processing.

Moreover, in the medical image processing control system 11, by acquiring and tabulating information on what kind of function the output source has, it is possible to grasp a possible image processing function by the output source and to automatically turn on the image processing function when a medical video source with the image processing function being valid is connected. That is, the medical image processing control system 11 can create a rule of always turning on a specific image processing function for a certain medical video source in advance, and create a table in which the rules are registered.

Furthermore, it is assumed that model names of a plurality of output sources and functions for each model are registered as presets in the medical image processing control system 11. Note that, in a case where there is no preset, in a case where the output source includes a function not registered in the preset, or the like, it is possible to turn off the function of automatically turning on the image processing function in this manner, or to turn on/off according to customization by the user.

FIG. 3 is a diagram illustrating an example of a control screen displayed on the operation terminal 32.

On the control screen 51 illustrated in FIG. 3, a thumbnail display unit 61a of the surgical site image, a thumbnail display unit 61b of the endoscopic image, a thumbnail display unit 61c of the vital image, and a thumbnail display unit 61d of the CT image are arranged along the upper side. Furthermore, a display control GUI 62-1 of the multifunctional medical monitor 28, a display control GUI 62-2 of the medical monitor 29, and a display control GUI 62-3 of the general monitor 30 are arranged along the left side. Moreover, below the thumbnail display units 61a to 61d and on the right side of the display control GUIs 62-1 to 62-3, display selection icons 63a-1 to 63d-3 are arranged at respective intersections.

Thumbnails of the respective images are displayed on the thumbnail display unit 61a of the surgical site image, the thumbnail display unit 61b of the endoscopic image, the thumbnail display unit 61c of the vital image, and the thumbnail display unit 61d of the CT image. In the display control GUI 62-1 of the multifunctional medical monitor 28, the display control GUI 62-2 of the medical monitor 29, and the display control GUI 62-3 of the general monitor 30, the picture-in-picture function can be turned on/off. For example, in the display control GUI 62-1 of the multifunctional medical monitor 28, the picture-in-picture function is turned on, and in the display control GUI 62-2 of the medical monitor 29 and the display control GUI 62-3 of the general monitor 30, the picture-in-picture function is turned off.

The display selection icons 63a-1 to 63d-3 are graphical user interfaces (GUIs) operated when any one of the surgical site image, the endoscopic image, the vital image, and the CT image is selected as the image to be displayed on the multifunctional medical monitor 28, the medical monitor 29, and the general monitor 30. Then, among the display selection icons 63a-1 to 63d-3, the colors of the display selection icons 63 corresponding to the images selected to be displayed on the multifunctional medical monitor 28, the medical monitor 29, and the general monitor 30 are changed and displayed (In the illustrated example, hatching is applied).

For example, the display selection icons 63a-1 to 63d-1 are operated when selecting an image to be displayed on the entire screen of the multifunctional medical monitor 28. In the illustrated example, the display selection icon 63d-1 is hatched to indicate a state in which it is selected to display the CT image on the entire screen of the multifunctional medical monitor 28. Furthermore, the display selection icons 63a-1P to 63d-1P are operated when selecting an image to be displayed on a picture-in-picture of the multifunctional medical monitor 28. In the illustrated example, the display selection icon 63c-1P is hatched, and a state in which displaying of a vital image on a picture-in-picture of the multifunctional medical monitor 28 is selected is illustrated.

Furthermore, the display selection icons 63a-2 to 63d-2 are operated when selecting an image to be displayed on the entire screen of the medical monitor 29. In the illustrated example, the display selection icon 63a-2 is hatched to indicate a state in which it is selected to display the surgical site image on the entire screen of the medical monitor 29. Then, the display selection icons 63a-3 to 63d-3 are operated when selecting an image to be displayed on the entire screen of the general monitor 30. In the illustrated example, the display selection icon 63b-3 is hatched to indicate a state in which it is selected to display the endoscopic image on the entire screen of the general monitor 30.

In this manner, the control screen 51 can grasp which medical video source is output to which output source by changing the color of the display selection icon 63 arranged at the intersection between the thumbnail display units 61a to 61d and the display control GUIs 62-1 to 62-3.

For example, the color of the display selection icon 63 is changed according to the image processing setting associated with the combination of the medical video source and the output source. Alternatively, a mark corresponding to the image processing setting may be displayed on the display selection icon 63, or the shape of the display selection icon 63 may be changed. Thus, each image processing setting can be easily recognized on the control screen 51.

Furthermore, on the control screen 51, when a specific operation is performed on the display selection icon 63, a status screen displaying image processing settings associated with a combination of the medical video source and the output source is displayed. Then, on the status screen, the image processing setting can be changed, and for example, the image processing setting can be changed to an image processing setting customized according to the preference of a surgeon who performs surgery using the medical image processing control system 11. For example, the image processing settings associated with the combination of the medical video source and the output source can be selected for each surgeon, and the customized image processing settings can be collectively changed.

### <Processing Example of Image Processing Setting Processing>

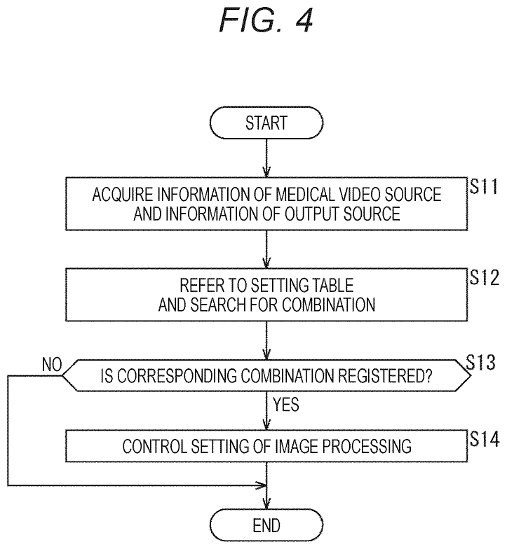

Image processing setting processing performed in the medical image processing control system 11 will be described with reference to a flowchart illustrated in FIG. 4.

For example, when the medical image processing control system 11 is activated, the processing is started, and in step S11, the acquisition unit 41 acquires information of a medical video source and information of an output source included in the medical image processing control system 11 via the IP switcher 26. Then, the acquisition unit 41 supplies the information of the medical video source and the information of the output source to the determination unit 42.

In step S12, the determination unit 42 refers to the setting table stored in the storage unit 31 on the basis of the information supplied from the acquisition unit 41 in step S11, and searches for the setting information of the combination of the medical video source and the output source.

In step S13, the determination unit 42 determines whether or not the setting information of the corresponding combination is registered in the setting table on the basis of the search result in step S12.

In a case where it is determined in step S13 that the setting information of the corresponding combination is registered in the setting table, the determination unit 42 notifies the control unit 43 that the setting information of the combination is registered in the setting table, and the processing proceeds to step S14.

In step S14, the control unit 43 reads the setting information notified from the determination unit 42 from the setting table. Then, the control unit 43 controls the setting (on/off) of the image processing to be applied as a whole of the medical image processing control system 11 according to the setting information for each combination of the medical video source and the output source. Then, after the processing of step S14 or in a case where it is determined in step S13 that the setting information is not registered in the setting table, the processing is terminated.

As described above, by performing the image processing setting processing, the medical image processing control system 11 can perform display of an image on which image processing suitable for a combination of the medical video source and the output source even when the output destination of the medical video source is switched.

Note that, in the medical image processing control system 11, the image processing setting can be prioritized or the image processing setting can be customized. For example, in a case where there are a main medical video source and output source and a sub medical video source and output source, and the processing power of the image processing control server 33 is insufficient to apply the rotation filter to both of them, the image processing settings can be customized to apply the rotation filter to the main medical video source and output source and not to apply the rotation filter to the sub medical video source and output source.

Furthermore, the medical image processing control system 11 can be customized to turn on or off image processing even for the same combination of the medical video source and the output source depending on the surgeon who uses it. For example, the medical image processing control system 11 can have a preset for each user and can store image processing settings for each combination of a medical video source and an output source depending on the surgeon performing the surgery using the medical image processing control system 11.

Moreover, the medical image processing control system 11 may set the priority of the image processing setting by combining any of the surgical method, patient information, lamp setting (shadowless light), and customization for each user, or may add a combination of the medical video source and the output source. For example, even in the same combination of the same medical video source and the output source, it is possible to add an image processing setting to turn off image processing functions such as the sharpening filter and the luminance enhancement filter when the shadowless light is set to warm color, and to turn on the image processing functions such as the sharpening filter and the luminance enhancement filter when the shadowless light is set to cool color.

Furthermore, in a case where the medical image processing control system 11 performs image analysis and grasps that gamma correction is not applied to the endoscopic image, or in a case where it can be determined that the image is dark, the medical image processing control system can add image processing setting to turn on gamma correction. For example, for an endoscopic image using an electric scalpel, image processing settings can be added so that the image processing functions such as the sharpening filter and the luminance enhancement filter are turned off, and the rotation filter is turned off when the menu screen is open. That is, when the rotation filter is applied while the menu screen is opened in the endoscopic image, a situation in which the menu screen is shaken or correction of the endoscopic image is not normally performed is assumed, and such a situation can be avoided.

Note that, in the medical image processing control system 11, for example, when an operation means is connected to the output-side IP converter 27 for control, the setting can be changed on the basis of the situations of the medical video source and the output source. For example, it is possible to control the output-side IP converter 27 to designate a region to be emphasized in the image processing functions such as the sharpening filter and the luminance enhancement filter.

Moreover, the medical image processing control system 11 can prohibit the change of the image processing setting or output an alert when an operation to change the image processing setting is performed depending on the combination of the medical video source and the output source. For example, in a case where there is an item prohibited from being set in a certain output source in a specific medical video source, the image processing setting can be prevented from being changed to such a setting.

Furthermore, in the medical image processing control system 11, the image processing setting can be fixed in a main output source, and the image processing setting can be automatically applied in a sub output source. Furthermore, in the medical image processing control system 11, in a case where the picture-in-picture function is turned on, it is preferable to turn off all pieces of image processing or to apply a filter before a picture-in-picture is generated. Alternatively, the image processing setting may be finely set according to a combination of two medical video sources and one output source that are targets of picture-in-picture.

Note that the medical image processing control system 11 may change the setting of gamma correction in the output source, may change the setting of gamma correction in the output-side IP converter 27, or may change the setting of gamma correction in the image processing control server 33.

### <Configuration Example of Computer>

Next, a series of processing (image processing method) described above can be performed by hardware or by software. In a case where the series of processing is performed by software, a program constituting the software is installed on a general-purpose computer, or the like.

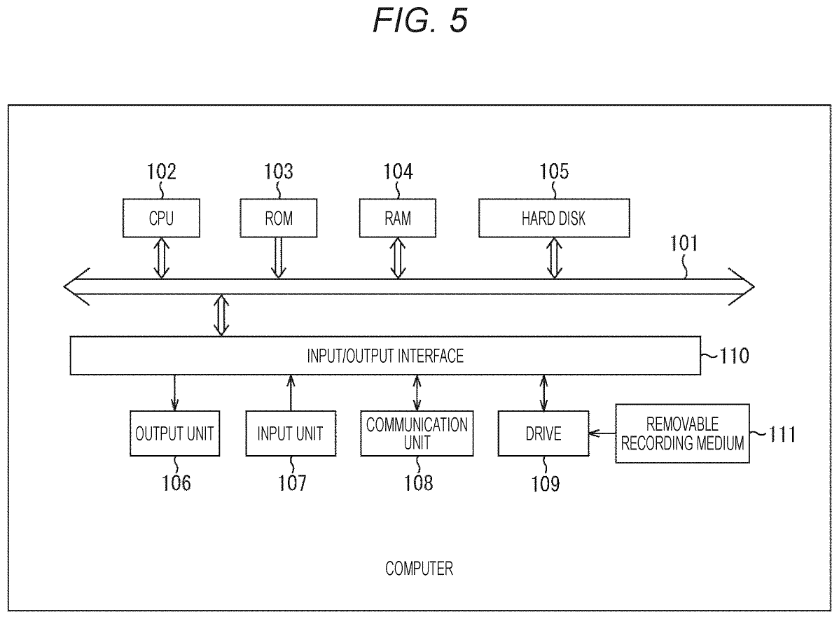

FIG. 5 is a block diagram illustrating a configuration example of an embodiment of a computer on which a program for executing the series of processing described above is installed.

The program can be recorded in advance on a hard disk 105 or a ROM 103 as a recording medium built in the computer.

Alternatively, further, the program can also be stored (recorded) in a removable recording medium 111 driven by a drive 109. Such a removable recording medium 111 can be provided as so-called package software. Here, examples of the removable recording medium 111 include, for example, a flexible disk, a compact disc read only memory (CD-ROM), a magneto optical (MO) disk, a digital versatile disc (DVD), a magnetic disk, a semiconductor memory, and the like.

Note that, in addition to installing the program on the computer from the removable recording medium 111 as described above, the program can be downloaded to the computer through a communication network or a broadcasting network and installed on the hard disk 105 to be incorporated. In other words, for example, the program can be wirelessly transferred from a download site to the computer through an artificial satellite for digital satellite broadcasting, or can be transferred by a wire to the computer through a network such as a local area network (LAN) and the Internet.

The computer has a built-in central processing unit (CPU) 102, and an input/output interface 110 is connected to the CPU 102 through a bus 101.

Upon receiving a command input by the user, for example, by operating an input unit 107 through the input/output interface 110, the CPU 102 executes a program stored in the read only memory (ROM) 103 accordingly. Alternatively, the CPU 102 loads a program stored in the hard disk 105 into a random access memory (RAM) 104 to execute the program.

Therefore, the CPU 102 performs processing according to the above-described flowchart or processing to be performed according to the above configuration described with the block diagram. Then, as necessary, the CPU 102 outputs a processing result from an output unit 106, or transmits the processing result from a communication unit 108, and further, causes the hard disk 105 to record the processing result, and the like, through the input/output interface 110, for example.

Note that, the input unit 107 includes a keyboard, a mouse, a microphone, and the like. Furthermore, the output unit 106 includes a liquid crystal display (LCD), a speaker, and the like.

Here, in the present description, the processing to be performed by the computer in accordance with the program is not necessarily performed in time-series according to the orders described in the flowcharts. That is, the processing to be performed by the computer in accordance with the program includes processing to be executed in parallel or independently of one another (parallel processing or object-based processing, for example).

Furthermore, the program may be processed by one computer (one processor) or processed in a distributed manner by a plurality of computers. Moreover, the program may be transferred to a distant computer to be executed.

Moreover, in the present description, a system means a set of a plurality of components (devices, modules (parts), and the like), and it does not matter whether or not all the components are in the same housing. Therefore, a plurality of devices housed in separate housings and connected to each other via a network and one device in which a plurality of modules is housed in one housing are both systems.

Furthermore, for example, a configuration described as one device (or one processing unit) may be divided and configured as the plurality of the devices (or the processing units). Conversely, configurations described above as a plurality of devices (or processing units) may be collectively configured as one device (or processing unit). Furthermore, it goes without saying that a configuration other than the above-described configurations may be added to the configuration of each device (or each processing unit). Moreover, when the configuration and operation as the entire system are substantially the same, a part of the configuration of a certain device (or processing unit) may be included in the configuration of another device (or another processing unit).

Furthermore, for example, the present technology can be configured as cloud computing in which one function is shared and jointly processed by the plurality of the devices through the network.

Furthermore, for example, the program described above can be executed by any device. In this case, the device is only required to have a necessary function (functional block and the like) and obtain necessary information.

Furthermore, for example, each step described in the flowcharts described above can be executed by one device, or can be executed in a shared manner by the plurality of the devices. Moreover, in a case where a plurality of pieces of processing is included in one step, the plurality of pieces of processing included in the one step can be executed by one device or executed by a plurality of devices in a shared manner. In other words, a plurality of pieces of processing included in one step can be executed as processing of a plurality of steps. Conversely, the processing described as the plurality of the steps can also be collectively executed as one step.

Note that, in the program to be executed by the computer, the processing in steps describing the program may be executed in time series in the order described in the present description, or may be executed in parallel, or independently at a necessary timing such as when a call is made. That is, as long as there is no contradiction, the processing of each step may be executed in an order different from the above-described order. Moreover, this processing of steps describing the program may be executed in parallel with processing of another program, or may be executed in combination with processing of another program.

Note that, a plurality of the present technologies that has been described in the present description can each be implemented independently as a single unit unless there is a contradiction. Of course, any plurality of the present technologies can also be used and implemented in combination. For example, a part or all of the present technologies described in any of the embodiments can be implemented in combination with a part or all of the present technologies described in other embodiments. Furthermore, a part or all of any of the above-described present technologies can be implemented together with another technology that is not described above.

### <Combination Examples of Configurations>

Note that the present technology can also have the following configurations.

(1)

An image processing control system including:
a plurality of medical video sources connected via a network;
a plurality of output sources connected via a network;
a switching unit that switches an output destination of the medical video sources to one of a plurality of the output sources; and
an image processing control unit that controls an image processing setting for performing display of an image on which image processing suitable for a combination of the medical video sources and the output sources has been applied even when the output destination of the medical video sources is switched.
   (2)

The image processing control system according to (1) above, in which
the image processing control unit includes
an acquisition unit that acquires information of the medical video sources and information of the output sources,
a determination unit that refers to a setting table in which setting information of image processing to be applied in each of combinations is registered for each of combinations of a plurality of the medical video sources and a plurality of the output sources on a basis of the information acquired by the acquisition unit, and determines whether or not the setting information of a corresponding combination is registered in the setting table, and
a control unit that controls a setting of the image processing according to the setting information in a case where the determination unit determines that the setting information of the corresponding combination is registered in the setting table.
   (3)

The image processing control system according to (1) or (2) above, in which
the image processing is performed in the output sources.
   (4)

The image processing control system according to (3) above, in which
the image processing control unit further includes an image processing unit that performs the image processing, and
on/off of the image processing in the output sources and the image processing in the image processing unit is set by the image processing setting.
   (5)

The image processing control system according to (4) above, in which
the image processing is image processing of emphasizing display of a blood vessel, and
in a case where both the output sources and the image processing unit includes an image processing function of emphasizing display of a blood vessel, the image processing setting is performed in such a manner that one image processing function is turned on and another image processing function is turned off.
   (6)

The image processing control system according to (4) above, in which
the image processing is a rotation filter that corrects rotation of an endoscopic image, and
in a case where an output destination of the endoscopic image is switched, the rotation filter is continuously applied to the output source after the switching.
   (7)

The image processing control system according to any one of (1) to (6) above, further including:
an operation terminal on which an operation of selecting an output destination of the medical video sources is performed, in which
a graphical user interface (GUI) for selecting the output source as an output destination of the medical video sources is displayed on a display unit of the operation terminal.
   (8)

The image processing control system according to (7) above, in which
display of the GUI is changed according to the image processing setting according to a combination of the medical video sources and the output sources.
   (9)

An image processing method including, by an image processing control system:
controlling, even when an output destination of a plurality of medical video sources connected via a network is switched to one of a plurality of output sources connected via the network, an image processing setting for performing display of an image on which image processing suitable for a combination of the plurality of medical video sources and the plurality of output sources connected has been performed.
   (10)

A program for causing a computer of an image processing control system to execute:
controlling, even when an output destination of a plurality of medical video sources connected via a network is switched to one of a plurality of output sources connected via the network, an image processing setting for performing display of an image on which image processing suitable for a combination of the plurality of medical video sources and the plurality of output sources connected has been performed.

Note that, the present embodiment is not limited to the embodiment described above, and various modifications can be made without departing from the gist of the present disclosure. Furthermore, the effects described in the present description are merely examples and are not limited, and other effects may be provided.

### REFERENCE SIGNS LIST

11 Medical image processing control system
21 Vital device
22 Endoscope
23 CT examination device
24 Surgical site camera
25 Input-side IP converter
26 IP switcher
27 Output-side IP converter
28 Multifunctional medical monitor
29 Medical monitor
30 General Monitor
31 Storage unit
32 Operation terminal
33 Image processing control server
41 Acquisition unit
42 Determination unit
43 Control unit
44 Image processing unit

1. An image processing control system comprising:
a plurality of medical video sources connected via a network;
a plurality of output sources connected via a network;
a switching unit that switches an output destination of the medical video sources to one of a plurality of the output sources; and
an image processing control unit that controls an image processing setting for performing display of an image on which image processing suitable for a combination of the medical video sources and the output sources has been applied even when the output destination of the medical video sources is switched.

2. The image processing control system according to claim 1, wherein
the image processing control unit includes
an acquisition unit that acquires information of the medical video sources and information of the output sources,
a determination unit that refers to a setting table in which setting information of image processing to be applied in each of combinations is registered for each of combinations of a plurality of the medical video sources and a plurality of the output sources on a basis of the information acquired by the acquisition unit, and determines whether or not the setting information of a corresponding combination is registered in the setting table, and
a control unit that controls a setting of the image processing according to the setting information in a case where the determination unit determines that the setting information of the corresponding combination is registered in the setting table.

3. The image processing control system according to claim 1, wherein
the image processing is performed in the output sources.

4. The image processing control system according to claim 3, wherein
the image processing control unit further includes an image processing unit that performs the image processing, and
on/off of the image processing in the output sources and the image processing in the image processing unit is set by the image processing setting.

5. The image processing control system according to claim 4, wherein
the image processing is image processing of emphasizing display of a blood vessel, and
in a case where both the output sources and the image processing unit includes an image processing function of emphasizing display of a blood vessel, the image processing setting is performed in such a manner that one image processing function is turned on and another image processing function is turned off.

6. The image processing control system according to claim 4, wherein
the image processing is a rotation filter that corrects rotation of an endoscopic image, and
in a case where an output destination of the endoscopic image is switched, the rotation filter is continuously applied to the output source after the switching.

7. The image processing control system according to claim 1, further comprising:
an operation terminal on which an operation of selecting an output destination of the medical video sources is performed, wherein
a graphical user interface (GUI) for selecting the output source as an output destination of the medical video sources is displayed on a display unit of the operation terminal.

8. The image processing control system according to claim 7, wherein
display of the GUI is changed according to the image processing setting according to a combination of the medical video sources and the output sources.

9. An image processing method comprising, by an image processing control system:
controlling, even when an output destination of a plurality of medical video sources connected via a network is switched to one of a plurality of output sources connected via the network, an image processing setting for performing display of an image on which image processing suitable for a combination of the plurality of medical video sources and the plurality of output sources connected has been performed.

10. A program for causing a computer of an image processing control system to execute:
controlling, even when an output destination of a plurality of medical video sources connected via a network is switched to one of a plurality of output sources connected via the network, an image processing setting for performing display of an image on which image processing suitable for a combination of the plurality of medical video sources and the plurality of output sources connected has been performed.
